(19) United States
(12) Patent Application Publication
(10) Pub. No.: US 2015/0184232 A1
(43) Pub. Date: Jul. 2, 2015

(54) MONITORING OF 1,4-DIOXANE BIODEGRADATION IN VARIOUS ENVIRONMENTS

(71) Applicants: Mengyan Li, Houston, TX (US); Jacques Mathieu, Houston, TX (US); Pedro J. Alvarez, Houston, TX (US)

(72) Inventors: Mengyan Li, Houston, TX (US); Jacques Mathieu, Houston, TX (US); Pedro J. Alvarez, Houston, TX (US)

(73) Assignee: William Marsh Rice University, Houston, TX (US)

(21) Appl. No.: 14/562,517

(22) Filed: Dec. 5, 2014

Related U.S. Application Data
(60) Provisional application No. 61/912,304, filed on Dec. 5, 2013.

(51) Int. Cl. C12Q 1/68

(52) U.S. Cl. CPC C12Q 1/689; C12Q 2600/158

(57) ABSTRACT

In some embodiments, the present disclosure pertains to methods of monitoring dioxane biodegradation in an environment by: (1) exposing a sample from the environment to an oligonucleotide probe that targets at least one bacterial nucleotide sequence; (2) detecting the presence of the at least one bacterial nucleotide sequence in the sample from the environment; and (3) correlating the presence of the at least one bacterial nucleotide sequence to dioxane biodegradation in the environment. In some embodiments, the methods of the present disclosure can be used to determine whether monitored natural attenuation (MNA) of dioxane will occur in the environment. In some embodiments, the methods of the present disclosure can be used to determine whether dioxane decontamination is needed. Additional embodiments of the present disclosure pertain to oligonucleotide probes for monitoring dioxane biodegradation in an environment.

### CROSS-REFERENCE TO RELATED APPLICATIONS

This application claims priority to U.S. Provisional Patent Application No. 61/912,304, filed on Dec. 5, 2013. The entirety of the aforementioned application is incorporated herein by reference.

### STATEMENT REGARDING FEDERALLY SPONSORED RESEARCH

This invention was made with government support under Strategic Environmental Research and Development Program (SERDP) Grant Number ER2301, awarded by the U.S. Department of Defense; and Grant Number W912HQ-13-C-0024, also awarded by the U.S. Department of Defense. The Government has certain rights in the invention.

### BACKGROUND

1,4-Dioxane (hereinafter referred to as “dioxane”) is a contaminant of emerging concern in numerous environments, such as environments impacted by chlorinated solvents. Current methods of detecting dioxane biodegradation in such environments have limitations, including limited sensitivity, limited specificity, and limited accuracy. As such, a need exists for improved methods of detecting dioxane biodegradation in various environments.

### BRIEF SUMMARY

In some embodiments, the present disclosure pertains to methods of monitoring dioxane biodegradation in an environment. In some embodiments, such methods include: (1) exposing a sample from the environment to an oligonucleotide probe that targets at least one bacterial nucleotide sequence; (2) detecting the presence of the at least one bacterial nucleotide sequence in the sample from the environment; and (3) correlating the presence of the at least one bacterial nucleotide sequence to dioxane biodegradation in the environment. Additional embodiments of the present disclosure pertain to oligonucleotide probes for monitoring dioxane biodegradation in an environment.

In some embodiments, the bacterial nucleotide sequence includes a bacterial DNA sequence. In some embodiments, the bacterial DNA sequence to be detected is derived from bacteria in the environment. In some embodiments, the bacterial DNA sequence spans or is near one or more genes involved in dioxane biodegradation. In some embodiments, the one or more genes fully or partially encode one or more tetrahydrofuran/dioxane monooxygenases. In some embodiments, the one or more genes include, without limitation, thmA, dxmA, and combinations thereof.

In some embodiments, the bacterial nucleotide sequence includes a bacterial RNA sequence. In some embodiments, the bacterial RNA sequence includes mRNA. In some embodiments, the mRNA is a full or partial transcript of one or more genes involved in dioxane biodegradation, such as thmA, dxmA, and combinations thereof. In some embodiments, the mRNA is a full or partial transcript of a tetrahydrofuran/dioxane monooxygenase.

In some embodiments, the oligonucleotide probes that target the bacterial nucleotide sequences include a plurality of oligonucleotides. In some embodiments, the oligonucleotide probe includes an oligonucleotide chemically conjugated to a fluorophore and quencher. In some embodiments, the oligonucleotide probe includes: a forward primer; a reverse primer; and a probe. In some embodiments, the forward primer is 5′-CTG TAT GGG CAT GCT TGT-3′ (SEQ ID NO: 1), the reverse primer is 5′-CCA GCG ATA CAG GTT CAT C-3′ (SEQ ID NO: 2), and the probe is 5′-(X)-ACG CCT ATT-(Y)-ACA TCC AGC AGC TCG A-(Z)-3′ (SEQ ID NO: 3). In some embodiments, X is a fluorophore that includes, without limitation, carboxy fluorescin (6-FAM), carboxyfluorescein diacetate succinimidyl ester (CFDA-SE), carboxyfluorescein succinimidyl ester (CFSE), cyanine dyes (e.g., Cy3 and Cy5), hexachlorofluorescein (HEX), and combinations thereof. In some embodiments, Y and Z are each quenchers that include, without limitation, TAMRA™ quencher dye, QSY® quencher, Black Hole Quencher® (BHQ), ZEN™ double-quenched probes (ZEN), IABkFQ, and combinations thereof. In some embodiments, X is carboxy fluorescin (6-FAM), Y is a ZEN™ double-quenched probe (ZEN), and Z is IABkFQ.

In some embodiments, bacterial nucleotide sequences are detected by amplification of the nucleotide sequence. In some embodiments, the amplification of the bacterial nucleotide sequence occurs by a polymerase chain reaction (PCR), such as real-time PCR or quantitative PCR.

In some embodiments, the detection of a bacterial nucleotide sequence occurs at different periods of time that span from about 1 hour to about 6 months. In some embodiments, an increase in the presence of the bacterial nucleotide sequence through a period of time is correlated to dioxane biodegradation in the environment. In some embodiments, the methods of the present disclosure can be used to determine whether monitored natural attenuation (MNA) of dioxane will occur in the environment. In some embodiments, the methods of the present disclosure can be used to determine whether dioxane decontamination is needed.

### BRIEF DESCRIPTION OF THE DRAWINGS

FIG. 1 provides a scheme of a method of detecting dioxane biodegradation in an environment.

FIG. 2 provides an illustration of a method of detecting dioxane biodegradation in an environment.

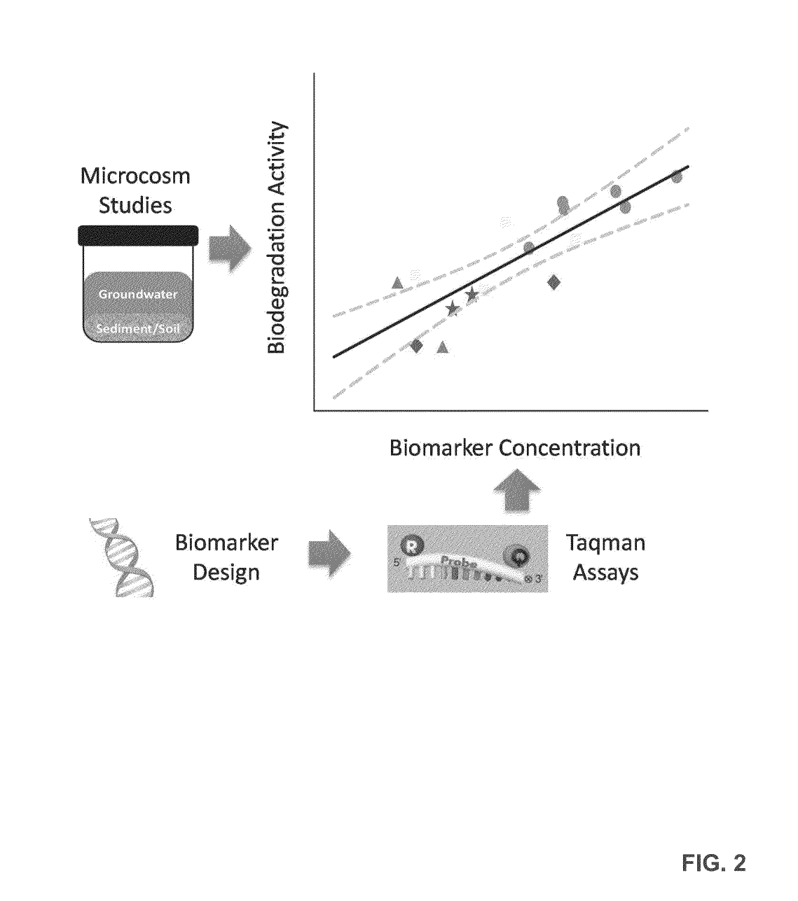

FIG. 3 shows the alignment of the deduced amino acid sequences corresponding to the large hydroxylase of soluble di-iron monooxygenases (SDIMOs) clustered by subfamily. The amino acid residues selected for biomarker design are highlighed in red and circled with rectangles. The conserved di-iron center (i.e., DE*RH) is highlighed in orange, and the hydrophobic residues surrounding the di-iron center are highlighted in blue. Numbers below the sequences correpsond to the numbering for dxmA from CB1190. Numbers in front of the subclusters represent the group numbers of SDIMOs. The bootstrapping neighbor-joining tree was generated by MEGA 5.2 using ClustalW as the computing algorithm.

FIG. 4 shows a correlation between the amount of consumed dioxane (μg) and the increase of thmA/dxmA gene copy numbers in microcosms on a normal (FIG. 4A) and a logarithmic (FIG. 4B) scale. The slope of the regression line of the left graph (FIG. 4A) was used to estimate the cell yield of dioxane for indigenous microbial degraders. The solid line represents the least square regression. The dashed lines represent the 95% confidence envelope.

FIG. 5 shows a correlation between zero-order dioxane biodegradation rates (μg/L/week) and final copy numbers of thmA/dxmA (FIG. 5A) but not 16S rRNA (FIG. 5B) genes in microcosms for various sites. The solid lines represent the least square regression. The dashed lines represent the 95% confidence envelope.

FIG. 6 provides quantitative polymerase chain reaction (Q-PCR) calibration curves for thmA/dxmA genes (FIG. 6A) and 16S rRNA (FIG. 6B).

FIG. 7 provides data relating to dioxane biodegradation in microcosms prepared with samples collected from sites located in the north slope of Alaska (A) and west Texas (T) and their corresponding sterile controls (marked as N).

FIG. 8 provides data relating to the detection of copy numbers of thmA genes in microcosms over three to five months' incubation. The red dot line represents the estimated value of the MDL. Double green dots indicate an increase with p value lower than 0.05. A single green dot indicates an increase with p value lower than 0.1.

FIG. 9 provides data relating to the detection of copy numbers of 16S rRNA genes in microcosms over three to five months' incubation. The red dot line represents the estimated value of the MDL. Double green dots indicate an increase with p value lower than 0.05. A single green dot indicates an increase with p value lower than 0.1. In contrast, double red dots indicate a decrease with p value lower than 0.05. A single red dot indicates a decrease with p value lower than 0.1.

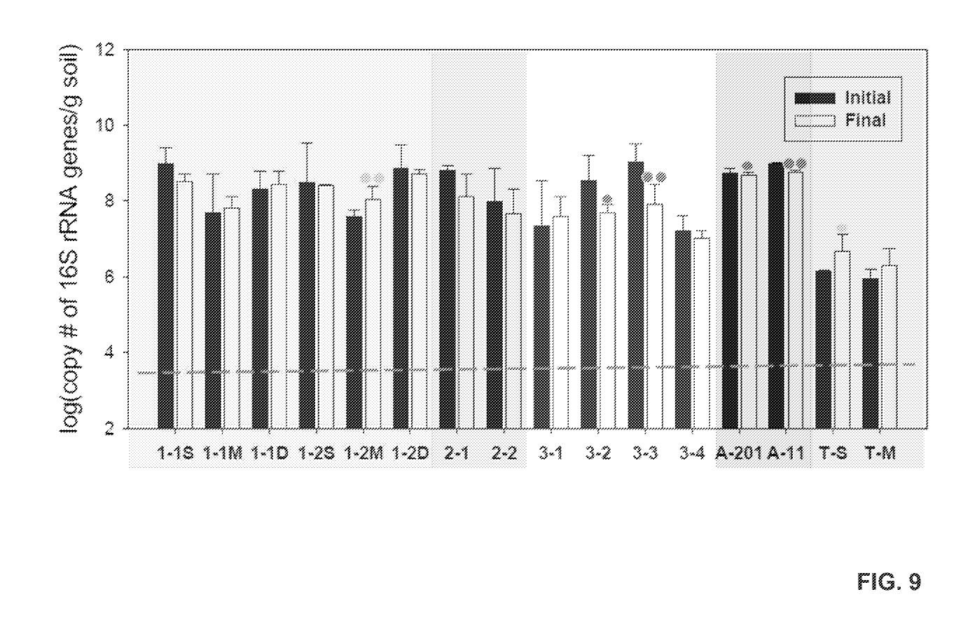

FIG. 10 provides a phylogenetic tree (constructed and visualized using MEGA 5.1) based on partial thmA/dxmA gene sequences from clone libraries constructed from soil samples collected in Microcosm 1-1S at week 20. Transformed clones were designated with numbers from 1 to 96. Trimmed sequences of four known thmA/dxmA genes were aligned to depict the evolutionary relationship, including dxmA from Pseudonocardia dioxanivorans CB 1190, thmA from Pseudonocardia tetrahydrofuranoxydans K1, and thmA from Pseudonocardia sp. ENV478, and thmA from Rhodococcus sp. YYL. The figure shows a high similarity between all DNA fragments amplified using the thmA/dxmA primer set in Example 1.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

Catabolic gene probes have been previously used to assess the capacity for biodegradation of a specific compound to occur at a contaminated site. 1,4-Dioxane (dioxane) is a groundwater contaminant of emerging concern due to its recently discovered widespread occurrence at thousands of sites impacted by chlorinated solvent releases, as well as its potential carcinogenicity. Dioxane was commonly used as a stabilizer for industrial solvents, typically 1,1,1-trichloroethane (1,1,1-TCA), thus explaining this common co-occurrence. Because of its recalcitrant cyclic ether structure and high mobility in aquifers, dioxane tends to impact large areas with low levels of contamination. In fact, dioxane represents a multi-billion dollar remediation challenge.

Monitored natural attenuation (MNA) is among the most cost-effective approaches to manage groundwater contamination by organic pollutants at low concentrations. However, the feasibility of MNA requires demonstration of site-specific biodegradation capabilities.

Recent findings by Applicants and others suggest that indigenous bacteria that can degrade dioxane might be more widespread than previously assumed. However, these studies relied on complex molecular biological techniques, such as cloning, microarray, restriction fragment length polymorphism (RFLP), and phospholipid fatty acid analysis associated with stable isotope probing, which can be labor-intensive and may not provide unequivocal evidence to link the abundance of the indigenous degraders to the intrinsic biodegradation activity.

As such, a need exists for more effective methods and probes for detecting dioxane biodegradation in an environment. The present disclosure addresses this need.

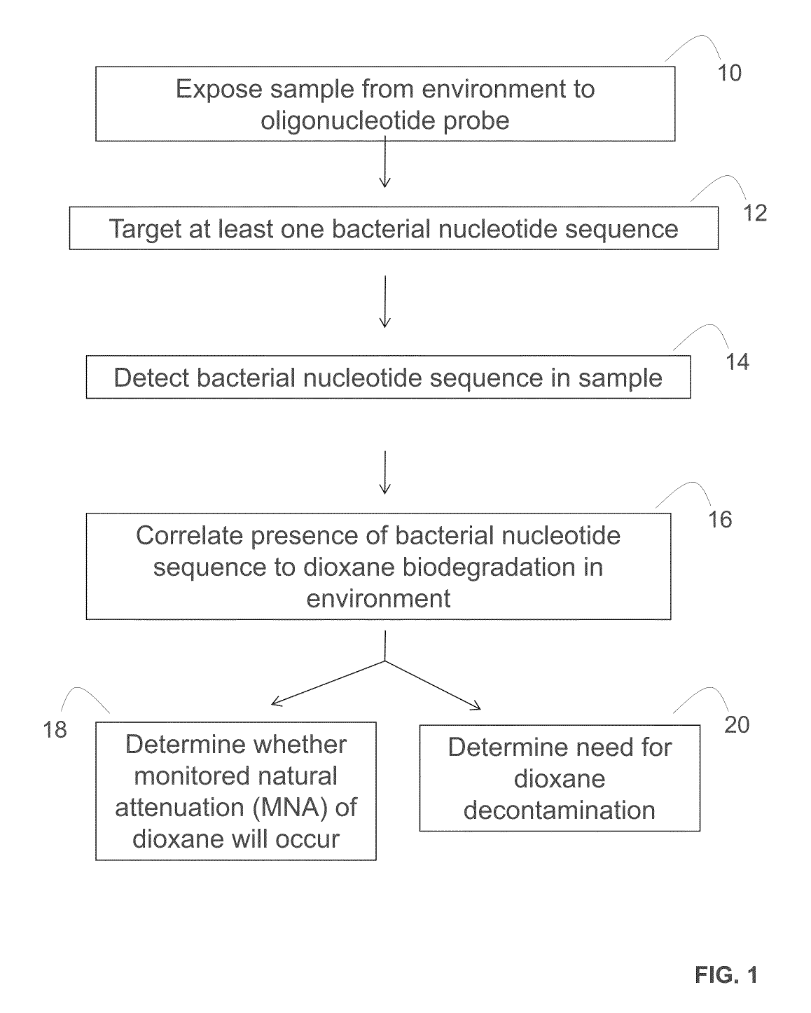

In some embodiments, the present disclosure pertains to methods of monitoring dioxane biodegradation in an environment. In some embodiments illustrated in FIG. 1, such methods include: exposing a sample from the environment to an oligonucleotide probe that targets at least one bacterial nucleotide sequence (steps 10 and 12); detecting the presence of the at least one bacterial nucleotide sequence in the sample from the environment (step 14); and correlating the presence of the at least one bacterial nucleotide sequence to dioxane biodegradation in the environment (step 16). In some embodiments, the methods of the present disclosure can be utilized to determine whether monitored natural attenuation (MNA) of dioxane will occur in the environment (step 18). In some embodiments, the methods of the present disclosure can be utilized to determine whether a need exists for dioxane decontamination in the environment (step 20).

Further embodiments of the present disclosure pertain to oligonucleotide probes for monitoring dioxane biodegradation in the environment. As set forth in more detail herein, the methods and oligonucleotide probes of the present disclosure can have numerous variations. For instance, various methods may be utilized to expose various types of samples from various environments to various oligonucleotide probes that target various bacterial nucleotide sequences. Moreover, various methods may be utilized to detect the presence of the bacterial nucleotide sequence in the environment and correlate its presence to dioxane biodegradation.

Environment

The methods of the present disclosure may be applied to various environments. For instance, in some embodiments, the environment is a dioxane-contaminated site. In some embodiments, the environment is a site impacted by chlorinated solvent release. In some embodiments, the environment includes, without limitation, aquifers, wells, groundwater wells, sludge tanks, landfills, and combinations thereof. In some embodiments, the environment is a dioxane-contaminated aquifer.

Exposing

Various methods may be used to expose the oligonucleotide probes of the present disclosure to a sample from an environment. In some embodiments, the exposing occurs by incubating the sample from the environment with the oligonucleotide probe. In some embodiments, the exposing occurs by incubating a sample from the environment (e.g., an aliquot) with the oligonucleotide probe. In some embodiments, the oligonucleotide probe is poured into a sample from an environment. In some embodiments, the oligonucleotide probe is sprayed onto a sample from an environment. Additional methods by which to expose the oligonucleotide probes of the present disclosure to a sample from an environment can also be envisioned.

Environmental Samples

The oligonucleotide probes of the present disclosure may be exposed to various types of samples from an environment. For instance, in some embodiments, the sample is an aliquot that is collected from an environment.

In some embodiments, the sample from the environment is unprocessed. In some embodiments, the sample from the environment is processed. For instance, in some embodiments, the sample from the environment includes bacterial nucleotides (e.g., bacterial DNA or bacterial RNA) that have been extracted from the bacteria in the environment. As such, additional embodiments of the present disclosure also include a step of extracting bacterial nucleotides (e.g., bacterial DNA or bacterial RNA) from a sample in an environment.

In some embodiments, the sample from the environment includes a sample that has been incubated (e.g., at room temperature) under aerobic conditions for a desired period of time (e.g., 30 minutes to 30 days). As such, additional embodiments of the present disclosure also include a step of incubating a sample from an environment under aerobic conditions for a desired period of time.

Bacterial Nucleotide Sequences

The oligonucleotide probes of the present disclosure may target various bacterial nucleotide sequences. In some embodiments, the bacterial nucleotide sequence includes a bacterial DNA sequence. In some embodiments, the bacterial DNA sequence is involved in dioxane biodegradation. In some embodiments, the bacterial DNA sequence spans or is near one or more genes involved in dioxane biodegradation. In some embodiments, the one or more genes include genes that encode one or more enzymes that initiate dioxane catabolism. In some embodiments, the one or more genes fully or partially encode one or more tetrahydrofuran/dioxane monooxygenases. In some embodiments, the one or more genes encode one or more subunits of tetrahydrofuran/dioxane monooxygenases. In some embodiments, the one or more genes encode a hydroxylase subunit of a tetrahydrofuran/dioxane monooxygenase. In some embodiments, the one or more genes encode the large hydroxylase subunits of tetrahydrofuran/dioxane monooxygenases. In some embodiments, the one or more genes include, without limitation, thmA, dxmA, and combinations thereof. In some embodiments, the one or more genes include thmA and dxmA.

In some embodiments, the bacterial DNA sequences that are targeted by the oligonucleotide probes of the present disclosure include conserved regions that span or are near one or more genes involved in dioxane biodegradation. In some embodiments, the bacterial DNA sequence includes a conserved region that spans or is near the active sites of one or more genes involved in dioxane biodegradation. In some embodiments, the bacterial DNA sequence includes a conserved region that spans or is near the active site of one or more genes encoding the large hydroxylase subunits of tetrahydrofuran/dioxane monooxygenases, such as thmA/dxmA genes.

In some embodiments, the bacterial nucleotide sequence includes a bacterial RNA sequence. In some embodiments, the bacterial RNA sequence includes mRNA. In some embodiments, the mRNA is a full or partial transcript of one or more genes involved in dioxane biodegradation (as previously described). In some embodiments, the mRNA is a full or partial transcript of a tetrahydrofuran/dioxane monooxygenase.

In some embodiments, the bacterial nucleotide sequence includes a bacterial cDNA sequence. In some embodiments, the bacterial cDNA sequence is derived from a bacterial mRNA.

The thmA/dxmA genes encode the large hydroxylase subunits of tetrahydrofuran (THF)/dioxane monooxygenases. Both genetic and enzymatic studies have indicated the vital role of THF/dioxane monooxygenases during the initial oxidation of cyclic ethers by bacteria. Moreover, the large hydroxylase subunits of THF/dioxane monooxygenases, which contain the active site, were found to be highly conserved (>97% identity) for the four bacteria known to metabolize the cyclic ethers THF and/or dioxane (i.e., Pseudonocardia dioxanivorans CB 1190, Pseudonocardia tetrahydrofuranoxydans K1, Pseudonocardia sp. ENV478, and Rhodococcus sp. YYL). In addition, the activity of THF/dioxane monooxygenases from CB1190 and K1 towards dioxane and THF has been verified by transformation and expression in a heterologous host, Rhodococcus jostii RHA1. Furthermore, microarray and denaturing gradient gel electrophoresis (DGGE) analyses demonstrated enrichment of thmA-like genes near the source zone of an Arctic dioxane-contaminated site, where the highest dioxane biodegradation activity was observed. As such, Applicants envision that bacterial DNA sequences that span or are near the thmA/dxmA genes, and mRNA transcripts of the thmA/dxmA genes, can be utilized in the present disclosure to monitor dioxane biodegradation.

In some embodiments, the bacterial nucleotide sequences (e.g., bacterial DNA sequences) that are targeted by the oligonucleotide probes of the present disclosure are derived from bacteria in the environment. In some embodiments, the bacterial nucleotide sequence is derived from bacteria that are indigenous in the environment. In some embodiments, the bacteria includes, without limitation, Pseudonocardia dioxanivorans CB 1190, Pseudonocardia tetrahydrofuranoxydans K1, Pseudonocardia sp. ENV478, Rhodococcus sp. YYL, Pseudomonas mendocina, Pseudomonas putida, Pseudomonas aeruginosa, Pseudomonas putida, Ralstonia pickettii, Burkholderia cepacia, Rhodococcus jostii, Methylomonas methanica, Escherichia coli, Nitrosomonas europaea, and combinations thereof.

Oligonucleotide Probes

The methods of the present disclosure may utilize various types of oligonucleotide probes to monitor dioxane biodegradation in various environments. Additional embodiments of the present disclosure pertain to such oligonucleotide probes.

In some embodiments, the oligonucleotide probes of the present disclosure include one or more oligonucleotides that target at least one bacterial nucleotide sequence of the present disclosure (as previously described). In some embodiments, the oligonucleotide probes of the present disclosure include one or more oligonucleotides that target at least one bacterial DNA sequence of the present disclosure (as previously described). In some embodiments, the oligonucleotide probes of the present disclosure include one or more oligonucleotides that target at least one bacterial RNA sequence of the present disclosure (as previously described).

In some embodiments, the oligonucleotide probes of the present disclosure include an oligonucleotide that is chemically conjugated to a fluorophore and a quencher. In some embodiments, the fluorophore includes, without limitation, carboxy fluorescin (6-FAM), carboxyfluorescein diacetate succinimidyl ester (CFDA-SE), carboxyfluorescein succinimidyl ester (CFSE), cyanine dyes (e.g., Cy3 and Cy5), hexachlorofluorescein (HEX), and combinations thereof. In some embodiments, the quencher includes, without limitation, TAMRA™ quencher dye, QSY® quencher, Black Hole Quencher® (BHQ), ZEN™ double-quenched probes (ZEN), IABkFQ, and combinations thereof.

In some embodiments, the oligonucleotide probes of the present disclosure include a plurality of oligonucleotides. In some embodiments, the oligonucleotide probes of the present disclosure include a forward primer, a reverse primer, and a probe. In some embodiments, the forward primer is 5′-CTG TAT GGG CAT GCT TGT-3′ (SEQ ID NO: 1). In some embodiments, the reverse primer is 5′-CCA GCG ATA CAG GTT CAT C-3′ (SEQ ID NO: 2). In some embodiments, the probe is 5′-(X)-ACG CCT ATT-(Y)-ACA TCC AGC AGC TCG A-(Z)-3′ (SEQ ID NO: 3), where X is a fluorophore, and where Y and Z are quenchers. In some embodiments, X is a fluorophore that includes, without limitation, carboxy fluorescin (6-FAM), carboxyfluorescein diacetate succinimidyl ester (CFDA-SE), carboxyfluorescein succinimidyl ester (CFSE), cyanine dyes (e.g., Cy3 and Cy5), hexachlorofluorescein (HEX), and combinations thereof. In some embodiments, X is carboxy fluorescin (6-FAM). In some embodiments, Y and Z are each quenchers that include, without limitation, TAMRA™ quencher dye, QSY® quencher, Black Hole Quencher® (BHQ), ZEN™ double-quenched probes (ZEN), IABkFQ, and combinations thereof. In some embodiments, Y is a ZEN™ double-quenched probe (ZEN), and Z is IABkFQ.

The oligonucleotide probes of the present disclosure may be synthesized in various manners. For instance, in some embodiments, the oligonucleotide probes of the present disclosure are synthesized using a DNA synthesizer. In some embodiments, the synthesized oligonucleotide probe is chemically conjugated to a fluorophore and quencher. In some embodiments, the components may be ordered from several vendors that specialize in the manufacture of DNA oligos. In some embodiments, the oligonucleotides of the present disclosure are designed for real-time quantitative PCR.

Bacterial Nucleotide Sequence Detection

Various methods may be utilized to detect bacterial nucleotide sequences in an environment. In some embodiments, the detecting includes amplification of the bacterial nucleotide sequence. In some embodiments, the amplification of the bacterial nucleotide sequence occurs by a polymerase chain reaction (PCR). In some embodiments, the amplification of the bacterial nucleotide sequence occurs by real-time PCR. In some embodiments, the amplification of the bacterial nucleotide sequence occurs by quantitative PCR, such as quantitative real-time PCR. In some embodiments where the bacterial nucleotide sequence is a bacterial RNA, the amplification can include reverse transcription PCR (RT-PCR).

In some embodiments, bacterial nucleotide sequences may be detected at a single period of time. In some embodiments, the detecting occurs at different periods of time. In some embodiments, the different periods of time are separated by hours, days, weeks, or months. In some embodiments, the detecting occurs at different periods of time that span from about 1 hour to about 6 months. In some embodiments, the different periods of time span from about 12 weeks to about 20 weeks. In some embodiments, the different periods of time span from about 3 months to about 6 months.

Correlation of Bacterial Nucleotide Presence to Dioxane Biodegradation

Various methods may also be utilized to correlate the presence of a bacterial nucleotide sequence to dioxane biodegradation. For instance, in some embodiments, an increase in the presence of at least one bacterial nucleotide sequence through a period of time is correlated to dioxane biodegradation in the environment. In some embodiments, the period of time spans for hours, days, weeks, or months. In some embodiments, the period of time spans from about 1 hour to about 6 months. In some embodiments, the period of time spans from about 3 months to about 5 months.

In some embodiments, a concentration of a detected bacterial nucleotide sequence is directly correlated to dioxane biodegradation activity. For instance, in some embodiments, the number of copies of nucleotide sequences are correlated to dioxane biodegradation activity. In some embodiments, the levels of the targeted nucleotide sequence are correlated with dioxane biodegradation activity. In some embodiments, the relative abundance of the targeted nucleotide sequence normalized to the total biomass in the environment is correlated with dioxane biodegradation activity.

Applications and Advantages

Currently, there are no probes available to assess dioxane biodegradation capacity. To Applicants' knowledge, the present disclosure provides the first methods and oligonucleotide probes for detecting dioxane biodegradation in various environments. Moreover, since there are estimates of hundreds of thousands of dioxane-impacted sites, the methods and oligonucleotide probes of the present disclosure may help a company save millions of dollars in potential clean-up costs associated with dioxane-contaminated sites. Furthermore, the methods of the present disclosure are relatively quick and inexpensive compared to current methods of detecting dioxane biodegradation.

As such, the methods and oligonucleotide probes of the present disclosure can find numerous applications. For instance, in some embodiments, the methods and oligonucleotide probes of the present disclosure can be used to determine whether monitored natural attenuation (MNA) of dioxane will occur in an environment. In some embodiments, the methods and oligonucleotide probes of the present disclosure can be used to determine whether dioxane decontamination is needed in an environment. In some embodiments, the methods and oligonucleotide probes of the present disclosure can be used to determine the level of dioxane decontamination that is needed in an environment.

MNA can occur when an indigenous microbial population in an environment degrades the contaminant of concern, thereby reducing the need for more expensive remediation treatments. Since the burden of proof lies with the proponent, it is necessary to support any claim of MNA with multiple, converging lines of evidence. As such, the methods and oligonucleotide probes of the present disclosure can also be used to provide such evidence in an accurate, effective and expedited manner. In some embodiments, the methods of the present disclosure may be one of the multiple lines of evidence that help prove the feasibility of MNA at a particular site.

Additional applications can also be envisioned. For instance, in some embodiments, the methods and oligonucleotide probes of the present disclosure can be used to monitor the performance of bioremediation treatments (e.g., biostimulation and bioaugmentation) at different dioxane-impacted sites. In some embodiments, such monitoring can occur world-wide. In some embodiments, the methods and oligonucleotide probes of the present disclosure can be used to evaluate the distribution and dynamics of dioxane-degrading microbes in the environment. In some embodiments, the methods and oligonucleotide probes of the present disclosure can be used to assess the dioxane biodegradation activity in activated sludge tanks that treat municipal and industrial (typically polyester plants) wastewater, as well as solid waste landfill leachate.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### EXAMPLE 1

### Correlation of Abundance of Tetrahydrofuran/Dioxane Monooxygenase Genes (thmA/dxmA) and 1,4-Dioxane Biodegradation at Various Impacted Aquifers

In this Example, a primer/probe set was developed to target bacterial genes encoding the large hydroxylase subunit of a putative tetrahydrofuran/dioxane monooxygenase (an enzyme proposed to initiate dioxane catabolism), using Taqman (5′-nuclease) chemistry. This effort relied on multiple sequence alignments of the four thmA/dxmA genes available on the NCBI database. The probe targets conserved regions surrounding the active site, thus enabling detection of multiple dioxane degraders. Real-time PCR using reference strain genomic DNA demonstrated the high selectivity (no false positives) and sensitivity of this probe (7,000˜8,000 copies/g soil). Microcosm tests prepared with groundwater samples from 16 monitoring wells at five different dioxane-impacted sites showed that enrichment of this catabolic gene (up to 114-fold) was significantly correlated to the amount of dioxane degraded. A significant correlation was also found between biodegradation rates and the abundance of thmA/dxmA genes. In contrast, 16S rRNA gene copy numbers (a measure of total bacteria) were neither sensitive nor reliable indicators of dioxane biodegradation activity. Overall, the results in this Example suggest that this novel catabolic biomarker (thmA/dxmA) has great potential to rapidly assess the performance of natural attenuation or bioremediation of dioxane plumes.

### EXAMPLE 1.1

### Primer and Probe Design

Multiple sequence alignment (Clustal X2.1, as described in Bioinformatics 2007, 23, (21), 2947-2948) was used to identify homologous regions between the four thmA/dxmA genes available on NCBI and avoid overlap with other soluble di-iron monooxygenase (SDIMO) genes that do not share the same primary substrate range. The phylogenetic tree based on amino acid sequences was then visualized using MEGA 5.1.

DNA residues 217 and 587 from the putative dxmA gene of CB 1190 were used as the input sequence for Primer Quest (Integrated DNA Technologies, Coralville, Iowa) to generate a series of possible primer/probe sets which satisfied the design criteria for TaqMan assays. After manual comparison and adjustment (Table 1), the final set was chosen allowing a nucleotide mismatch not greater than 1, including the forward primer, 5′-CTG TAT GGG CAT GCT TGT-3′ (SEQ ID NO: 1), the reverse primer, 5′-CCA GCG ATA CAG GTT CAT C-3′ (SEQ ID NO: 2), and the probe, 5′-(6-FAM)-ACG CCT ATT-(ZEN)-ACA TCC AGC AGC TCG A-(IABkFQ)-3′ (SEQ ID NO: 3).

TABLE 1
Properties of the primers and probe targeting thmA/dxmA genes.

| Probe/Primer | Name | Sequence (5′-3′) | Size | GC Content | Tm |
|---|---|---|---|---|---|
| Forward | thmA | CTG TAT GGG CAT GCT | 18 | 50 | 59.8 |
| Primer | TqFWD330 | TGT | | | |
| Reverse | thmA | CCA GCG ATA CAG GTT | 19 | 52.6 | 59.7 |
| Primer | TgREV444 | CAT C | | | |
| Taqman | thmA | /6-FAM/ACG CCT ATT | 25 | 52 | 68.9 |
| Probe | TqPRB377 | /ZEN/ACA TCC AGC AGC | | | |
| | | TCG A/IABkFQ/ | | | |

The amplicons were approximately 115 by in length. All primers and probes were synthesized by Integrated DNA Technologies, and a novel internal quencher ZEN was integrated to reduce background noise.

### EXAMPLE 1.2

### Specificity and Coverage Tests with Bacterial Genomic DNA

To evaluate the specificity and selectivity of the thmA/dxmA probe and primer set, qPCR was conducted with the genomic DNA isolated from reference strains (Table 2). After growth in LB or R2A media at room temperature for 1 to 7 days, cells were harvested by centrifugation, and their genomic DNA was extracted using an UltraClean Microbial DNA Isolation Kit (MoBio, Carlsbad, Calif.). The final DNA concentrations were measured by UV spectroscopy using an ND-1000 Spectrophotometer (NanoDrop, Wilmington, Del.).

TABLE 2
Specificity and coverage tests for the designed thmA/dxmA biomarker.

| Gene | Encoding | SDIMO | | Biomarker Detection^{b} | |
|---|---|---|---|---|---|
| Name | Enzymes^{a} | Group | Microorganism Strain | thmA/dxmA | 16S rRNA |
| dxm | Dioxane MO | 5 | Pseudonocardia | + | + |
| | | | dioxanivorans CB1190 | | |
| thm | Tetrahydrofuran | 5 | Pseudonocardia | + | + |
| | MO | | tetrahydrofuranoxydans | | |
| | | | K1 | | |
| tmo | Toluene-4-MO | 2 | Pseudomonas mendocina | − | + |
| | | | KR1 | | |
| tbu | Toluene-3-MO | 2 | Ralstonia pickettii PKO1 | − | + |
| tom | Toluene-2-MO | 1 | Burkholderia cepacia G4 | − | + |
| dmp | Phenol HD | 1 | Pseudomonas putida | − | + |
| | | | CF600 | | |
| prm | Propane MO | 5 | Rhodococcus jostii RHA1 | − | + |
| mmo | Soluble methane | 3 | Methylomonas methanica | − | + |
| | MO | | MC09 | | |
| — | — | — | Escherichia coli K12 | − | + |
| — | — | — | Bacteriophage λ | − | − |
| amo | Ammonia MO | — | Nitrosomonas europaea | − | + |
| | | | Winogradsky | | |
| tod | Toluene DO | — | Pseudomonas putida F1 | − | + |
| xyl | Toluate 1,2-DO | — | Pseudomonas aeruginosa | − | + |
| | | | PAO1 | | |

^{a}MO = monooxygenase; HD = hydroxylase; DO = dioxygenase.
^{b}+ indicates a positive detection was obtained above the detection limit by using the primers/probe set in qPCR; − indicates no positive detection was obtained above the detection limit by using the primers/probe set in qPCR.

### EXAMPLE 1.3

### Microcosm Studies

To assess the efficacy of the catabolic biomarker in enhancing the forensic analysis of monitored natural attenuation (MNA), aquifer materials and groundwater samples were collected from 20 monitoring wells from 5 different dioxane-impacted sites in the U.S. (3 in CA, 1 in AK, and 1 in TX). Triplicate microcosms were prepared with dioxane-impacted groundwater (100 to 150 mL with initial dioxane concentrations reaching up to 46,000 μg/L) and aquifer materials (50 g), and incubated at room temperature under aerobic conditions. To distinguish abiotic losses of dioxane, sterile controls were prepared with autoclaved samples and poisoned with HgCl₂ (200 mg/L). Dioxane concentrations were monitored for 12 to 20 weeks using a frozen micro-extraction method followed by GC/MS (Ground Water Monit R 2011, 31, (4), 70-76).

At the beginning and the termination of the microcosm experiments, 10 mL of sample mixture was transferred into a 15 mL centrifugation tube. Aquifer materials together with biomass were separated by centrifugation at ×10,000 g for 20 min. Total microbial genomic DNA was extracted using a PowerSoil DNA Isolation Kit (MoBio, Carlsbad, Calif.). The eluted DNA (100 μL) was further purified and concentrated to 16 μL using a Genomic DNA Clean & Concentrator Kit (Zymo Resesarch, Irvine, Calif.). The DNA Extraction efficiency and PCR inhibition factor were determined by recovery of bacteriophage λ DNA (Sigma-Aldrich, St. Louis, Mo.), which was added as internal standard.¹⁵

### EXAMPLE 1.4

### Quantitative PCR

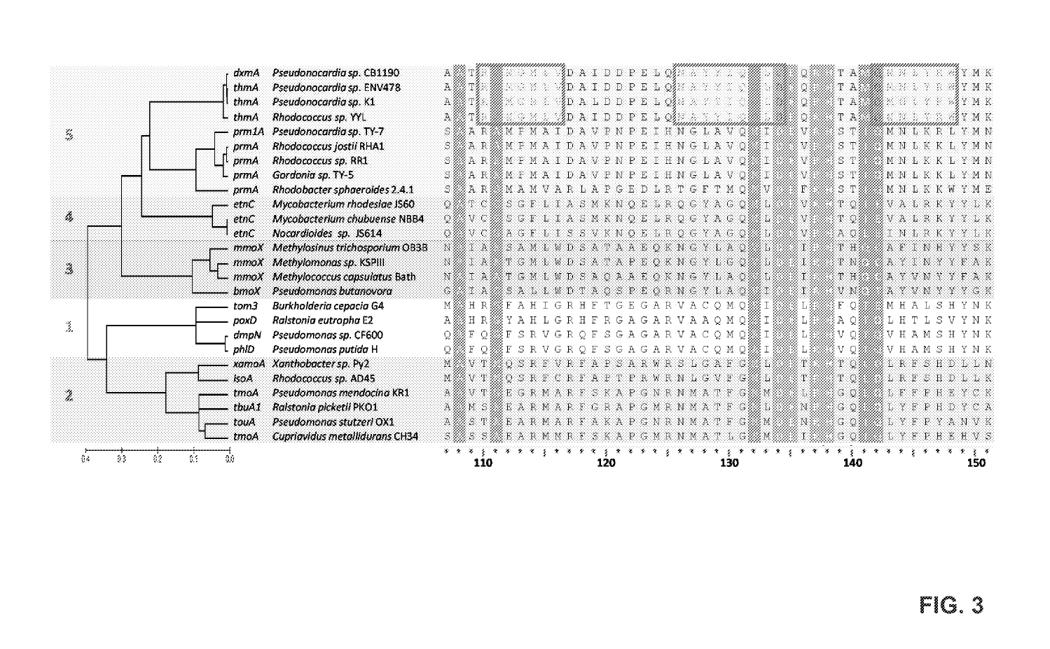

qPCR assisted with Taqman assays was used to quantify thmA/dxmA genes from dioxane-degrading bacteria as well as total Bacteria (Microbiol-Sgm 2002, 148, 257-266). The PCR reaction mixture contained 1 μL of undiluted DNA (or 1 ng/μL diluted bacterial genomic DNA), 300 nM of the forward and reverse primers, 150 nM of the fluorogenic probe, 10 μL of TaqMan universal master mix II (Applied Biosystems, Foster City, Calif.), and DNA-free water to reach a total volume of 20 μL. The qPCR was performed with a 7500 Real-Time PCR system (Applied Biosystems, Foster City, Calif.) using the following temperature program: 50° C. for 2 min, 95° C. for 10 min, and 40 cycles of 95° C. for 15 s and 60° C. for 1 min. Serial dilutions (10⁻⁴˜10¹ ng DNA/μL) of the extracted genomic DNA of CB 1190 were utilized to prepare the calibration curves for both thmA/dxmA (1 copy/genome) and 16S rRNA (3 copies/genome) genes (FIG. 3). Assuming a genome size of 7.44 Mb and 9.124×10¹⁴ bp/μg (i.e., [6.022×10¹⁷ Da/μg of DNA]/[660 Da/bp]) for CB1190, the gene copy numbers were calculated based on the equation below:

$$\frac{gene copies}{µL} = ( \frac{\frac{µg of DNA}{µL}}{\frac{7.44 Mb}{genome}} ) ( \frac{9.124 \times {10}^{14} bp}{µg of DNA} ) ( \frac{gene copies}{genome} )$$

Method detection limits (MDLs) were 7,000˜8,000 copies of thmA/dxmA genes/g soil and 2,000˜3,000 copies of 16S rRNA genes/g soil (Table 3).

TABLE 3
Method detection limits (MDLs) for
thmA/dxmA and 16S rRNA genes

| Parameter | thmA/dxmA | 16S rRNA |
|---|---|---|
| qPCR instrument MDL | 123 | 37 |
| (copy numbers/reaction mixture) | | |
| Overall MDL^{a} | 7,203-7,984 | 2,324-2,573 |
| (copy numbers/g soil) | | |

^{a}Including DNA recovery and an F of 64.

DNA extraction recoveries ranged from 2.3 to 48.9%. Similar recovery ranges are commonly reported for soil DNA extractions, with the lower values reflecting sequential elution and residual impurities that hinder Taq polymerase reactions.

### EXAMPLE 1.5

### The thmA/dxmA probe is selective

Biochemical, structural, and evolutionary studies indicate that the large hydroxylases of all the enzymes belonging to this SDIMO family contain a highly conserved carboxylate-bridged di-iron center (i.e., DE*RH motif) that serves as the active site for hydroxylation or peroxidation reactions (FIG. 3). However, different groups of SDIMOs exhibit different substrate specificity. Substrate recognition and binding may be primarily associated with the hydrophobic residues that surround the di-iron center, because these are conserved within each SDIMO group. Since only THF/dioxane monooxygenases are of interest in this Example, the criteria for the thmA/dxmA biomarker design consisted of i) avoiding the di-iron centers conserved by all SDIMOs, and ii) targeting the surrounding hydrophobic residues only shared by THF/dioxane monooxygenases.

FIG. 3 illustrates that the amino acid residues targeted by the thmA/dxmA primers/probe set are identical among all four known THF/dioxane monooxygenases, but significantly different from other SDIMOs. The q-PCR analysis indicated that both dxmA from CB1190 and thmA from K1 (which were the positive controls we had readily available) were detected with comparable sensitivity (C_{T} values approximately 25 for 1 ng genomic DNA). Negative controls, using genomic DNA from bacteria with other types of SDIMOs (e.g., bacteria with dioxygenases such as Pseudomonas putida F1, and bacteria without SDIMOs such as Escherichia coli K12), and bacteriophage λ, were used to assess the potential for false positives. None of these negative controls were detected by this thmA/dxmA probe and primer set, but previously designed primer sets by Applicants using the SYBR Green system had yielded false positives for other SDIMO genes (e.g., tmo), indicating that the use of TaqMan probes significantly reduces the possibility of hybridization with non-specific templates. These results corroborate that the thmA/dxmA probe and primer set Applicants developed for the TaqMan system enables sensitive detection of thm/dxm genes and avoids false positives from other oxygenase genes that bear a close evolutionary relationship.

### EXAMPLE 1.6

### Dioxane biodegradation activity was significantly correlated to thmA/dxmA abundance

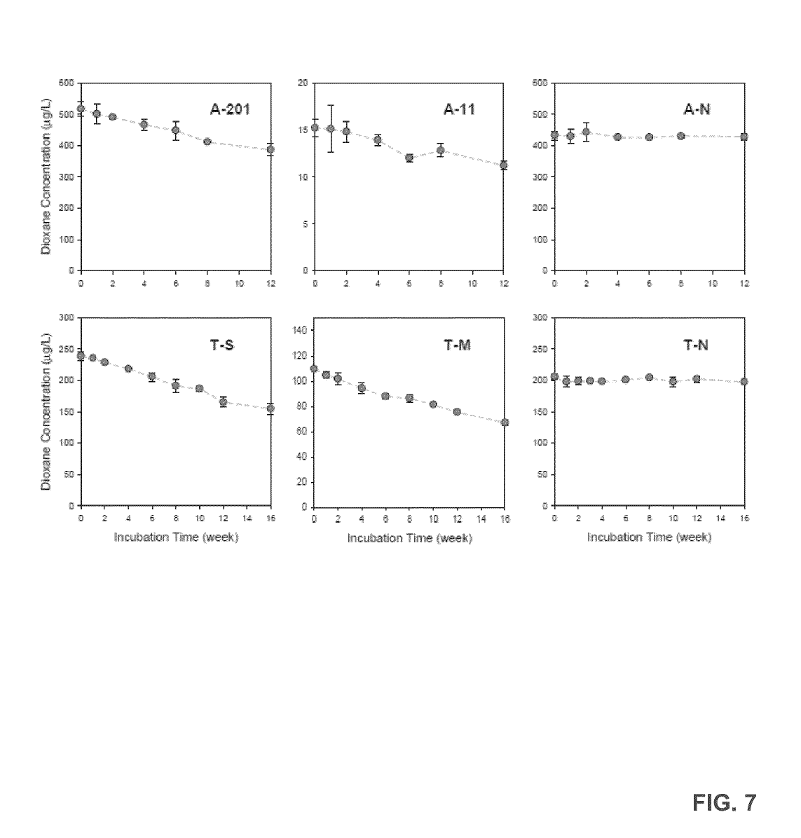

After three to five months of incubation, considerable dioxane removal was observed in 16 of the 20 microcosms compared to the sterile controls, indicating the presence of dioxane degraders at these sites (FIG. 7). The fitted zero-order decay rates varied from 10⁻¹ to 10³ μg/L/week (Table 4).

TABLE 4
Microcosm preparation and observed dioxane biodegradation rates.

| | | Distance | Initial Dioxane | Zero-order Dioxane |
|---|---|---|---|---|
| | Sampling | from the | Concentration | Degradation Rate |
| Site | Locations | Source (ft) | (μg/L) | (μg/L/week) |
| CA1 | 1-1S | 0 | 46049.8 ± 2429.7 | 3448.7 ± 459.3 |
| | 1-1M | 0 | 30906.1 ± 804.7 | 1548.7 ± 132.9 |
| | 1-1D | 0 | 14214 ± 920.2 | 654.2 ± 49.5 |
| | 1-2S | 200 | 1540.3 ± 103.9 | 69.6 ± 2.2 |
| | 1-2M | 200 | 12034.8 ± 319.4 | 584.2 ± 14.7 |
| | 1-2D | 200 | 19289.8 ± 1056.9 | 848.6 ± 51.6 |
| | 1-4 | 1550 | 412.7 ± 24.8 | — |
| | 1-5 | 3900 | 203.8 ± 12.8 | — |
| | 1-6 | NA | ND(≦1.6) | — |
| CA2 | 2-1 | NA | 248.2 ± 7.8 | 9.9 ± 0.8 |
| | 2-2 | NA | 7.5 ± 0.2 | 0.3 ± 0.1 |
| CA3 | 3-1 | 0 | 7150.7 ± 203.6 | 326.5 ± 8.5 |
| | 3-2 | 200 | 2261.5 ± 78.2 | 112.1 ± 5.6 |
| | 3-3 | 1000 | 876 ± 13.9 | 17.4 ± 3.2 |
| | 3-4 | 1350 | 582.9 ± 38.7 | 7.2 ± 0.9 |
| | 3-5 | NA | 32.4 ± 0.8 | — |
| AK | A-201 | 0 | 516.8 ± 23.7 | 11.1 ± 0.7 |
| | A-11 | 195 | 15.2 ± 0.9 | 0.4 ± 0.1 |
| TX | T-S | 0 | 238.6 ± 7.1 | 5.6 ± 0.2 |
| | T-M | 1700 | 109.7 ± 1.5 | 2.6 ± 0.1 |

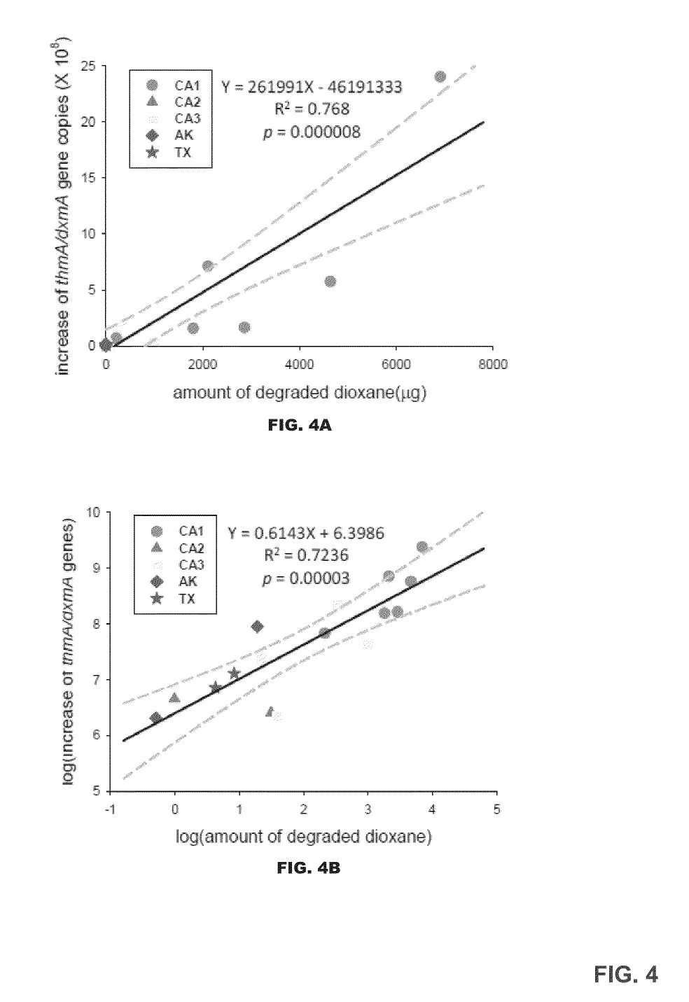

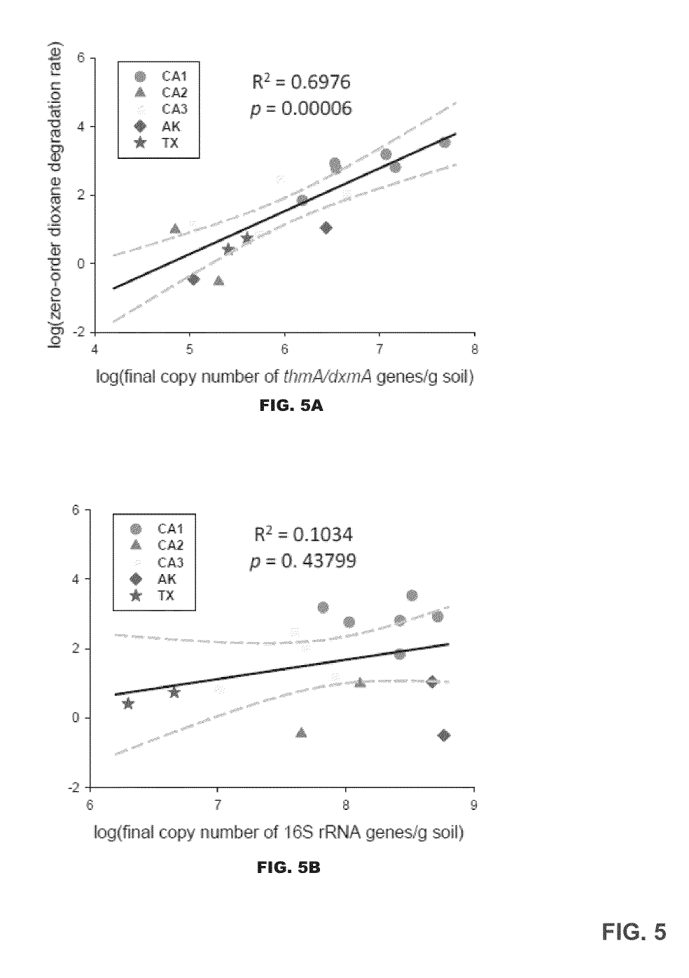

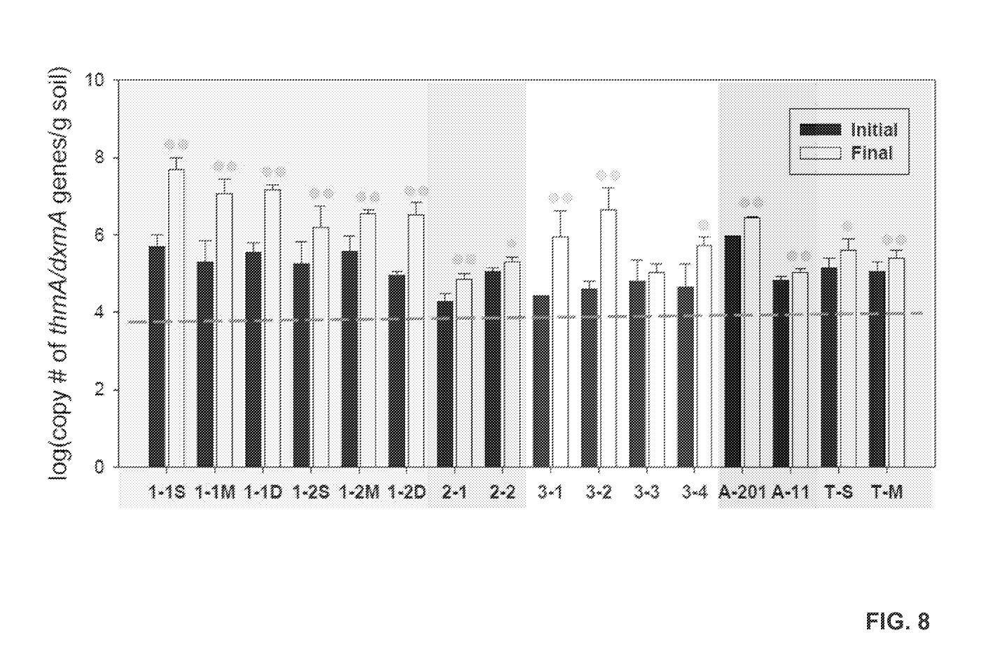

Growth of dioxane degraders was evident by an increase in thmA/dxmA copy numbers, up to 114-fold (FIG. 8). This increase was significantly correlated (p<0.05, R²=0.72) to the amount of consumed dioxane (FIGS. 4A-B). However, thmA/dxmA genes were not detected in killed controls or in microcosms prepared with background samples that did not experience dioxane removal (e.g., 1-6 and 3-5 in Table 4). Assuming a dry cell weight of 10⁻¹² g and protein composition of 55%, the cell yield coefficient (Y) for the indigenous dioxane degraders was estimated as 0.14 mg protein/mg dioxane (i.e., Y=ΔX/ΔS=regression line slope in FIG. 4A), which is comparable with reported yield coefficients for CB1190 (0.01˜0.09 mg protein/mg dioxane) and other dioxane metabolizers, such as Mycobacterium sp. D11 (0.18 mg protein/mg dioxane). A significant correlation (p<0.05, R²=0.70) was also observed between the final thmA/dxmA copy numbers and dioxane biodegradation rates (FIG. 5A). In contrast, copy numbers of 16S rRNA genes (a phylogenetic biomarker that is commonly used to enumerate total bacteria) were not significantly correlated (p=0.44) to dioxane biodegradation activity (FIG. 5B), corroborating the selectivity of this thmA/dxmA probe.

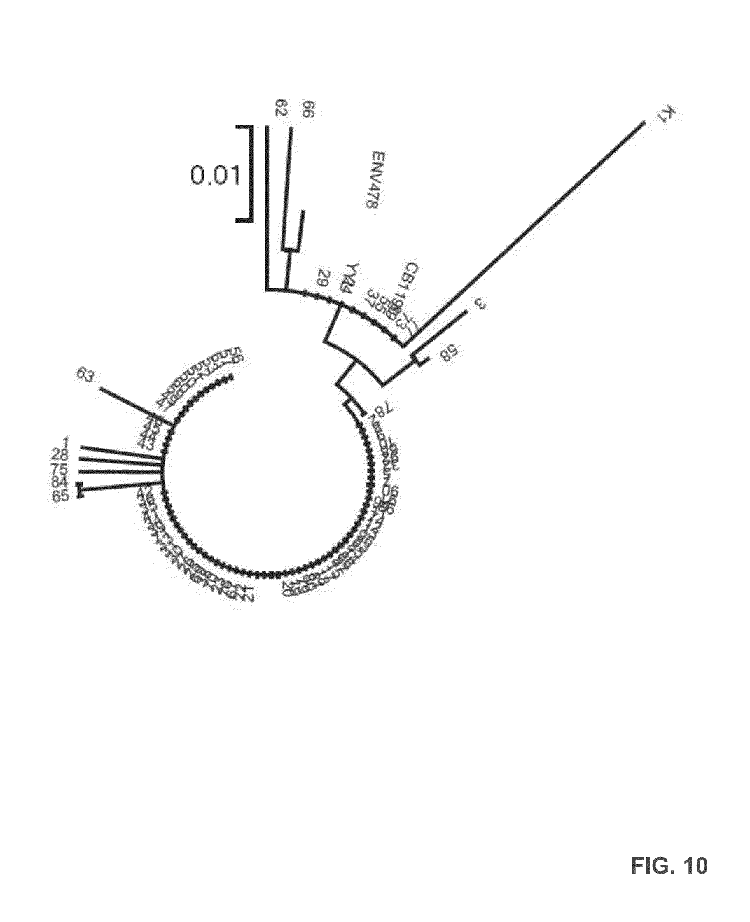

To further verify that amplification products from the complex environmental samples were actually fragments of the intended thmA/dxmA genes, a clone library was constructed with genomic DNA isolated from Microcosm 1-1S (Example 1.7-1.8), generating a total of 86 valid clones that were sequenced and aligned (FIG. 10). All sequences exhibited high identity with previously reported thmA/dxmA genes (≧95%), and no more than one nucleotide mismatch was found between the Taqman probe and its targeted sequences in the clone library, which provides further evidence for the reliability of the primer/probe set.

Applicants recognize that numerous site-specific factors could confound the correlation between biodegradation activity and thmA/dxmA abundance. These include nutrient and electron acceptor influx, redox conditions, pH, temperature and presence of inhibitory compounds. However, such confounding factors are likely to affect similarly both biodegradation rates and biomarker enrichment (through microbial growth or decay) over the large temporal scales that are relevant to MNA. Thus, these results suggest that thmA/dxmA can be a valuable biomarker to help determine the feasibility and assess the performance of MNA at dioxane-impacted sites.

### EXAMPLE 1.7

### Calibration Curves and Method Detection Limits (MDLs)

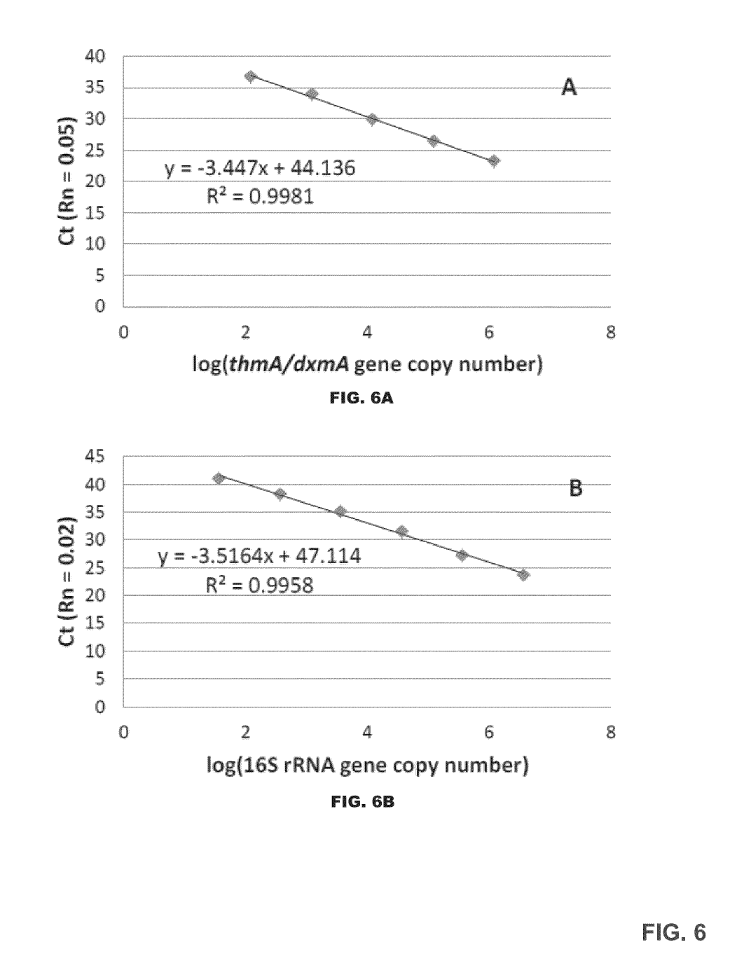

The calibration curves (FIG. 6) were generated using series dilution of standard CB1190 DNA samples (10-4-101 ng genomic DNA/pL) corresponding to a known gene copy number over six orders of magnitude (i.e., 12-1.23×10⁶ for thmA/dxmA and 37-3.68×10⁶ for 16S rRNA). High amplification efficiency of 95% was obtained in thmA/dxmA quantification, with an R₂ value of 0.998 and a slope of −3.45. Similarly, efficiency of 92% was obtained for quantification of 16S rRNA genes, with an R₂ value of 0.996 and a slope of −3.52.

The qPCR instrument MDL is identified as the minimum detectable copy number when seven sequential analyses were successful. The overall MDLs (in copy numbers/gram of aquifer materials in microcosms) are calculated as the instrument qPCR MDLs (in copy numbers/reaction mixture) adjusted with the DNA recoveries of these seven quantifications and the proportion (F) of the DNA used as the template in the qPCR.

$$Overall MCL = \frac{qPCR instrument MDL}{DNA Recovery} \times F$$

### EXAMPLE 1.8

### Clone Library Construction

PCR was performed in 50 μL samples with 1 μL concentrated genomic DNA, 0.2 mM dNTPs, 2.5 mM MgCl_{2,} 0.8 mM of each primer (Table 1), 1× Green GoTaq Flexi buffer, and 1.25 U GoTaq Hot Start polymerase (Promega, Madison, Wis.). Thermocycling conditions for the PCR reaction were as follows: initial denaturation at 95° C. for 5 min, followed by 30 cycles of 95° C. for 45 s, 50° C. for 45 s, and 73° C. for 30 s, and a final elongation at 73° C. for 5 min. The final PCR products (approximately 115 bp) were checked by gel electrophoresis.

The PCR products were then purified and concentrated using DNA Clean & Concentrator-5 kit (Zymo Research, Irvine, Calif.), and TOPO cloned into the pCR4-TOPO TA vector using the TOPO TA Cloning kit for Sequencing (Invitrogen, Carlsbad, Calif.) according to the manufacturer's instructions. The TOPO reaction mixture was transformed into TOP10 competent cells (Invitrogen, Carlsbad, Calif.), which were grown on LB agar plates containing the antibiotic ampicillin. A total of 96 colonies were picked and cultured in LB medium with 50 μg/mL ampicillin overnight. Plasmid DNA was prepped using a proprietary alkaline lysis protocol followed by ethanol precipitation. DNA cycle sequencing was performed using BigDye Terminator v3.1 chemistry in conjunction with the M13F universal primer. Sequencing reactions were cleaned up on Sephadex. Sequence delineation and base calling were performed using an ABI model 3730 XL automated fluorescent DNA sequencer by SeqWright Genomic Services (Houston, Tex.).

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of monitoring dioxane biodegradation in an environment, said method comprising:
exposing a sample from the environment to an oligonucleotide probe,
wherein the oligonucleotide probe comprises one or more oligonucleotides that target at least one bacterial nucleotide sequence;
detecting presence of the at least one bacterial nucleotide sequence in the sample from the environment; and
correlating the presence of the at least one bacterial nucleotide sequence to dioxane biodegradation in the environment.

2. The method of claim 1, wherein the exposing comprises incubating the sample from the environment with the oligonucleotide probe.

3. The method of claim 1, wherein the sample from the environment comprises extracted bacterial nucleotides.

4. The method of claim 1, wherein the environment is selected from the group consisting of aquifers, wells, groundwater wells, sludge tanks, landfills, and combinations thereof.

5. The method of claim 1, wherein the bacterial nucleotide sequence comprises a bacterial DNA sequence.

6. The method of claim 5, wherein the bacterial DNA sequence spans or is near one or more genes involved in dioxane biodegradation.

7. The method of claim 6, wherein the one or more genes fully or partially encode one or more tetrahydrofuran/dioxane monooxygenases.

8. The method of claim 6, wherein the one or more genes are selected from the group consisting of thmA, dxmA, and combinations thereof.

9. The method of claim 1, wherein the bacterial nucleotide sequence comprises a bacterial RNA sequence.

10. The method of claim 9, wherein the bacterial RNA sequence comprises mRNA, wherein the mRNA is a full or partial transcript of one or more genes involved in dioxane biodegradation.

11. The method of claim 10, wherein the one or more genes are selected from the group consisting of thmA, dxmA, and combinations thereof.

12. The method of claim 10, wherein the mRNA is a full or partial transcript of a tetrahydrofuran/dioxane monooxygenase.

13. The method of claim 1, wherein the bacterial nucleotide sequence is derived from bacteria in the environment.

14. The method of claim 1, wherein the oligonucleotide probe comprises an oligonucleotide chemically conjugated to a fluorophore and a quencher.

15. The method of claim 14, wherein the fluorophore is selected from the group consisting of carboxy fluorescin (6-FAM), carboxyfluorescein diacetate succinimidyl ester (CFDA-SE), carboxyfluorescein succinimidyl ester (CFSE), cyanine dyes, hexachlorofluorescein (HEX), and combinations thereof.

16. The method of claim 14, wherein the quencher is selected from the group consisting of TAMRA™ quencher dye, QSY® quencher, Black Hole Quencher® (BHQ), ZEN™ double-quenched probes (ZEN), IABkFQ, and combinations thereof.

17. The method of claim 1, wherein the oligonucleotide probe comprises a plurality of oligonucleotides.

18. The method of claim 17, wherein the oligonucleotide probe comprises:
a forward primer;
a reverse primer; and
a probe.

19. The method of claim 18, wherein the forward primer is 5′-CTG TAT GGG CAT GCT TGT-3′ (SEQ ID NO: 1).

20. The method of claim 18, wherein the reverse primer is 5′-CCA GCG ATA CAG GTT CAT C-3′ (SEQ ID NO: 2).

21. The method of claim 18, wherein the probe is 5′-(X)-ACG CCT ATT-(Y)-ACA TCC AGC AGC TCG A-(Z)-3′ (SEQ ID NO: 3),
wherein X is a fluorophore, and wherein Y and Z are each quenchers.

22. The method of claim 21,
wherein X is a fluorophore selected from the group consisting of carboxy fluorescin (6-FAM), carboxyfluorescein diacetate succinimidyl ester (CFDA-SE), carboxyfluorescein succinimidyl ester (CFSE), cyanine dyes, hexachlorofluorescein (HEX), and combinations thereof; and
wherein Y and Z are each quenchers selected from the group consisting of TAMRA™ quencher dye, QSY® quencher, Black Hole Quencher® (BHQ), ZEN™ double-quenched probes (ZEN), IABkFQ, and combinations thereof.

23. The method of the claim 1, wherein the detecting comprises amplification of the bacterial nucleotide sequence.

24. The method of claim 23, wherein the amplification of the bacterial nucleotide sequence occurs by a polymerase chain reaction (PCR).

25. The method of claim 24, wherein the amplification of the bacterial nucleotide sequence occurs by real-time PCR.

26. The method of claim 24, wherein the amplification of the bacterial nucleotide sequence occurs by quantitative PCR.

27. The method of claim 1, wherein the detecting occurs at different periods of time.

28. The method of claim 27, wherein the different periods of time span from about 1 hour to about 6 months.

29. The method of claim 1, wherein an increase in the presence of the at least one bacterial nucleotide sequence through a period of time is correlated to dioxane biodegradation in the environment.

30. The method of claim 29, wherein the period of time spans from about 1 hour to about 6 months.

31. The method of claim 1, wherein the method is used to determine whether monitored natural attenuation (MNA) of dioxane will occur in the environment.

32. The method of claim 1, wherein the method is used to determine whether dioxane decontamination is needed.

33. An oligonucleotide probe for monitoring dioxane biodegradation in an environment, wherein the oligonucleotide probe comprises:
a forward primer comprising the sequence 5′-CTG TAT GGG CAT GCT TGT-3′ (SEQ ID NO: 1);
a reverse primer comprising the sequence 5′-CCA GCG ATA CAG GTT CAT C-3′ (SEQ ID NO: 2); and
a probe.

34. The oligonucleotide probe of claim 33, wherein the probe comprises an oligonucleotide chemically conjugated to a fluorophore and a quencher.

35. The oligonucleotide probe of claim 34, wherein the fluorophore is selected from the group consisting of carboxy fluorescin (6-FAM), carboxyfluorescein diacetate succinimidyl ester (CFDA-SE), carboxyfluorescein succinimidyl ester (CFSE), cyanine dyes, hexachlorofluorescein (HEX), and combinations thereof.

36. The oligonucleotide probe of claim 34, wherein the quencher is selected from the group consisting of TAMRA™ quencher dye, QSY® quencher, Black Hole Quencher® (BHQ), ZEN™ double-quenched probes (ZEN), IABkFQ, and combinations thereof.

37. The oligonucleotide probe of claim 33, wherein the probe comprises 5′-(X)-ACG CCT ATT-(Y)-ACA TCC AGC AGC TCG A-(Z)-3′ (SEQ ID NO: 3),
wherein X is a fluorophore, and
wherein Y and Z are each quenchers.

38. The oligonucleotide probe of claim 37, wherein X is a fluorophore selected from the group consisting of carboxy fluorescin (6-FAM), carboxyfluorescein diacetate succinimidyl ester (CFDA-SE), carboxyfluorescein succinimidyl ester (CFSE), cyanine dyes, hexachlorofluorescein (HEX), and combinations thereof.

39. The oligonucleotide probe of claim 37, wherein Y and Z are each quenchers selected from the group consisting of TAMRA™ quencher dye, QSY® quencher, Black Hole Quencher® (BHQ), ZEN™ double-quenched probes (ZEN), IABkFQ, and combinations thereof.

40. The oligonucleotide probe of claim 37, wherein X is carboxy fluorescin (6-FAM), wherein Y is a ZEN™ double-quenched probe (ZEN), and wherein Z is IABkFQ.


SEQUENCE LISTING (3 sequences)

SEQ ID NO: 1 - DNA, 18 bp, Artificial Sequence
  Synthetic Oligonucleotide
ctgtatgggcatgcttgt

SEQ ID NO: 2 - DNA, 19 bp, Artificial Sequence
  Synthetic Oligonucleotide
ccagcgatacaggttcatc

SEQ ID NO: 3 - DNA, 25 bp, Artificial Sequence
  Synthetic Oligonucleotide
acgcctattacatccagcagctcga